(19) United States
(12) Patent Application Publication
(10) Pub. No.: US 2016/0151212 A1
(43) Pub. Date: Jun. 2, 2016

(54) Male Incontinence Assembly

(71) Applicant: Juliet R. McCatty, Brooklyn, NY (US)

(72) Inventor: Juliet R. McCatty, Brooklyn, NY (US)

(21) Appl. No.: 14/557,568

(22) Filed: Dec. 2, 2014

(51) Int. Cl. A61F 13/491; A61F 13/72; A61F 13/505

(52) U.S. Cl. CPC A61F 13/4915; A61F 13/505; A61F 13/72; A61F 2013/15121

(57) ABSTRACT

A male incontinence assembly includes an inner covering positionable against genitals of a person. The inner covering includes a panel that is flexible and has an inner surface and an outer surface. The panel has a front edge and a rear edge. The panel is formable into a wearable position such that the inner surface is concavely arcuate from the front edge to the rear edge. A central portion positioned between the front and rear edges comprises a crotch area. An absorbent pad is mounted on the inner surface. An outer covering comprises a pair of shorts that is positionable around the inner covering. The outer covering completely covers the inner covering such that the inner covering is not viewable from a front side of the outer covering. The outer covering can be detached from the inner covering and the inner covering replaced if the inner covering becomes soiled.

### BACKGROUND OF THE DISCLOSURE

### Field of the Disclosure

The disclosure relates to incontinence devices and more particularly pertains to a new incontinence device which is not easily viewable to others.

### SUMMARY OF THE DISCLOSURE

An embodiment of the disclosure meets the needs presented above by generally comprising an inner covering configured for positioning against genitals of a person. The inner covering includes a panel that is flexible and has an inner surface and an outer surface. The panel has a front edge, a rear edge, a first side edge and a second side edge. The panel is formable into a wearable position such that the inner surface is concavely arcuate from the front edge to the rear edge. A central portion positioned between the front and rear edges comprises a crotch area. An absorbent pad is mounted on the inner surface and is spaced from the front and rear edges and is positioned on the crotch area. An outer covering comprises a pair of shorts that is positionable around the inner covering when the inner covering is in the wearable position such that a juncture of the first side edge and the front edge remains spaced from a juncture of the first side edge and the rear edge, and such that a juncture of the second side edge and the front edge remains spaced from a juncture of the second side edge and the rear edge. The outer covering completely covers the inner covering such that the inner covering is not viewable from a front side of the outer covering. The outer covering can be detached from the inner covering and the inner covering replaced if the inner covering becomes soiled.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF THE DRAWINGS

The disclosure will be better understood and objects other than those set forth above will become apparent when consideration is given to the following detailed description thereof. Such description makes reference to the annexed drawings wherein:

FIG. 1 is a front view of a male incontinence assembly according to an embodiment of the disclosure.

FIG. 2 is a top view of an embodiment of the disclosure.

FIG. 3 is front view of an embodiment of the disclosure.

FIG. 4 is a top view of an embodiment of the disclosure having an inner covering thereof removed and taken along line 4-4 of FIG. 3.

FIG. 5 is a cross-sectional view of an embodiment of the disclosure taken along line 5-5 of FIG. 4.

### DESCRIPTION OF THE PREFERRED EMBODIMENT

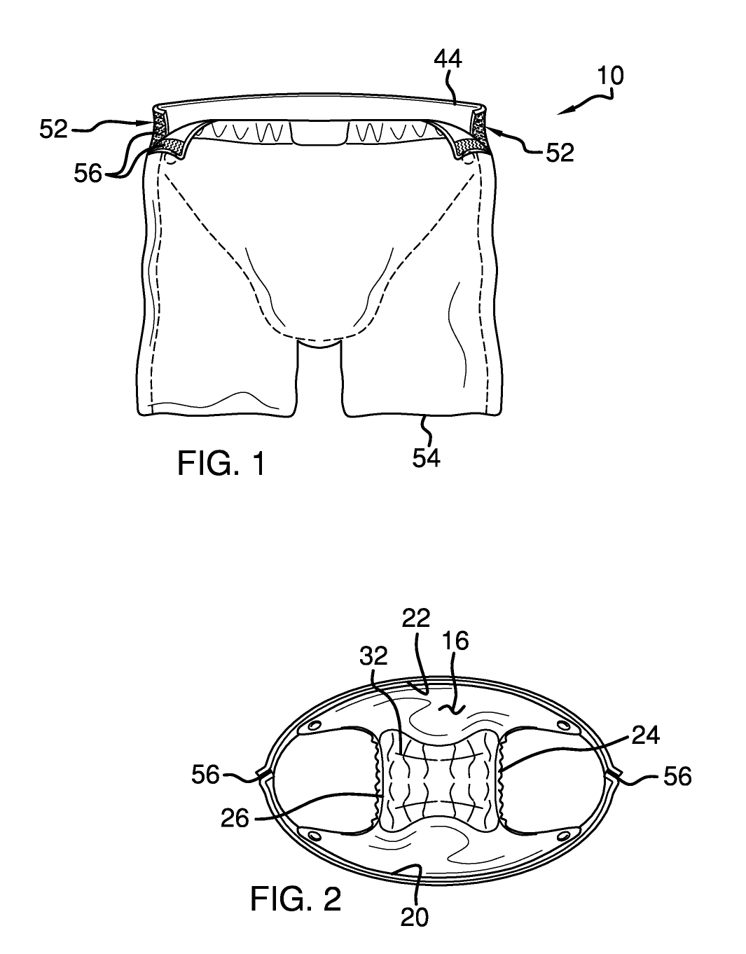

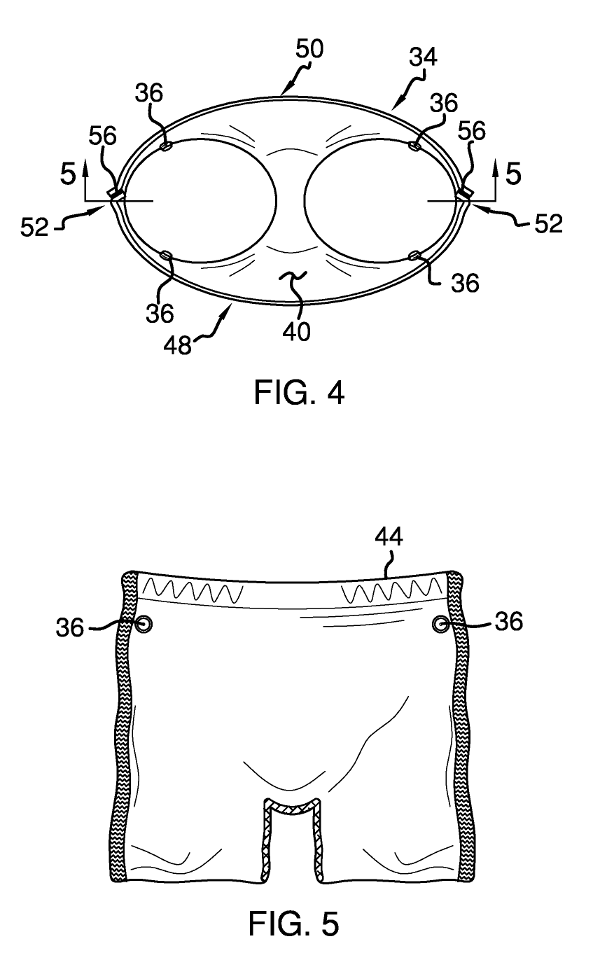

With reference now to the drawings, and in particular to FIGS. 1 through 5 thereof, a new incontinence device embodying the principles and concepts of an embodiment of the disclosure and generally designated by the reference numeral 10 will be described.

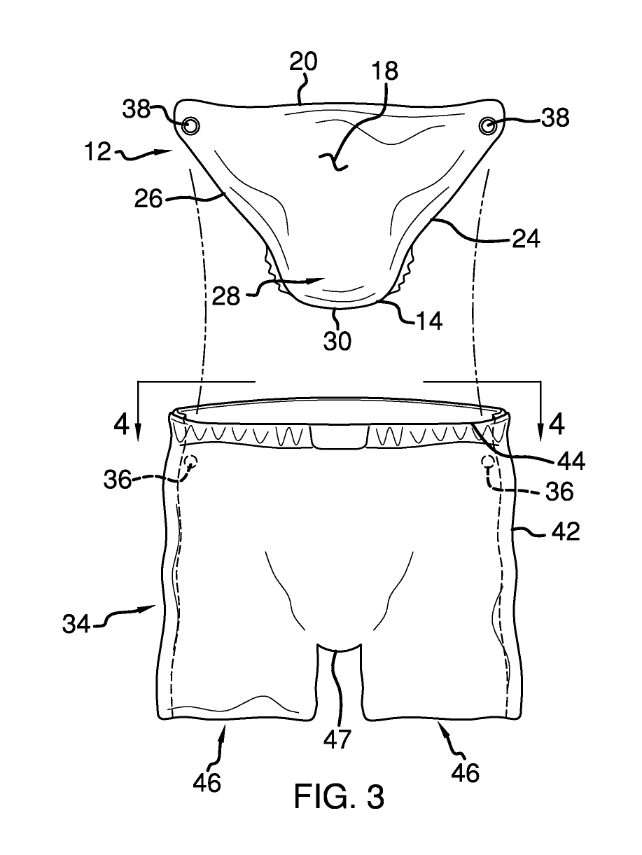

As best illustrated in FIGS. 1 through 5, the male incontinence assembly 10 generally comprises an inner covering 12 configured for positioning against genitals of a person. The inner covering 12 includes a panel 14 that is flexible and has an inner surface 16 and an outer surface 18. The panel 14 may be comprised of a fluid impermeable material. The panel 14 has a front edge 20, a rear edge 22, a first side edge 24 and a second side edge 26. Each of the first 24 and second 26 side edges is concavely arcuate to form around a person's legs. The panel 14 is formable into a wearable position, as shown in FIG. 3, such that the inner surface 16 is concavely arcuate from the front edge 20 to the rear edge 22. A central portion positioned between the front 20 and rear 22 edges comprises a crotch area 28. A lower most point of the outer surface 18 of the crotch area 28 defines a base 30 of the inner covering 12. An absorbent pad 32 is mounted on the inner surface 16 and may be non-removably attached thereto. The absorbent pad 32 is spaced from the front 20 and rear 22 edges and is positioned on the crotch area 28.

An outer covering 34 comprises a pair of shorts and may be made of a cloth material to resemble a conventional pair of boxer briefs. The outer covering 34 is positionable around the inner covering 12 when the inner covering 12 is in the wearable position such that a juncture of the first side edge and the front edge 20 remains spaced from a juncture of the first side edge 24 and the rear edge 22, and such that a juncture of the second side edge 26 and the front edge 20 remains spaced from a juncture of the second side edge 26 and the rear edge 22. The outer covering 34 completely covers the inner covering 12 such that the inner covering 12 is not viewable from a front side 48 of the outer covering 34 as is demonstrated in FIG. 1.

The outer covering 34 is removably coupled to the inner covering 12. This may be achieved in a plurality of manners. One way of achieving this, as shown in the Figures, is by way of snaps having first mating members 36 attached to the inner covering 12 and second mating members 38 attached to an inside surface 40 of the outer covering 34. The first 36 and second 38 mating members are positioned to align properly with each other. The outer covering 34 includes a perimeter wall 42 having an upper edge defining a waistband 44. The front 20 and rear 22 edges are each positioned adjacent to the waistband 44 and the first 36 and second 38 mating members are also positioned adjacent to the waistband 44.

The perimeter wall 42 extends downwardly from the waist band 44 and is formed into a pair of legs 46 with a closed area 47 therebetween which is positioned directly below the crotch area 28. Each of the legs 46 extends downwardly below the inner covering 12 a distance between 2.0 inches and 12.0 inches and this is measured vertically downward from a horizontal line intersecting the lower most point 30 of the outer surface 18. The outer covering 34 includes the front side 48 and the back side 50 and the outer covering 34 further includes a pair of breaks 52 therein each extending through the waistband 44 and through a lower edge 54 of the outer covering 34 such that each of the legs 46 is divided approximately into a front portion and a back portion. Thus the outer covering 34, when the breaks are opened, is held together by the closed area 47.

A pair of couplers 56 is provided to releasably close the breaks 52. Each of the breaks 52 has an edge having one of the couplers 56 attached thereto. The couplers 56 may each comprise a hook and loop coupler extending approximately along an entire length of an associated one of the breaks 52. The outer covering 34 can thusly be opened along the breaks 52 and then be detached from the inner covering 12.

In use, the assembly 10 is worn as would be a typical pair of boxer briefs. However, the inner covering 12 will capture and absorb and bodily fluids from the person wearing the assembly 10. If the absorbent pad 32 is soiled, the user may simply and detach the inner covering 12 from the outer covering 34, open the outer covering 34 along one or both of the breaks 52 to gain access to the inner covering 12 for its removal, and replace the inner covering 12 with a new one having non-soiled pad 32. The assembly 10 thus provides the ability to hide the fact that a person is wearing an incontinence device.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. An incontinence undergarment assembly comprising:
an inner covering configured for positioning against genitals of a person, said inner covering including a panel being flexible and having an inner surface and an outer surface, said panel having a front edge, a rear edge, a first side edge and a second side edge, said panel being formable into a wearable position such that said inner surface is concavely arcuate from said front edge to said rear edge, a central portion positioned between said front and rear edges comprising a crotch area;
an absorbent pad being mounted on said inner surface, said absorbent pad being spaced from said front and rear edges and being positioned on said crotch area;
an outer covering comprises a pair of shorts, said outer covering being positionable around said inner covering when said inner covering is in said wearable position such that a juncture of said first side edge and said front edge remains spaced from a juncture of said first side edge and said rear edge, and such that a juncture of said second side edge and said front edge remains spaced from a juncture of said second side edge and said rear edge, said outer covering completely covering said inner covering such that said inner covering is not viewable from a front side of said outer covering; and
wherein said outer covering can be detached from said inner covering and said inner covering replaced if said inner covering becomes soiled.

2. The assembly according to claim 1, wherein each of said first and second side edges is concavely arcuate.

3. The assembly according to claim 1, wherein said outer covering includes a perimeter wall having an upper edge defining a waistband, said perimeter wall extending downwardly from said waist band and being formed into a pair of legs, each of said legs extending downwardly below said inner covering a distance between 2.0 inches and 12.0 inches.

4. The assembly according to claim 3, wherein said outer covering includes said front side and said back side, said outer covering including a pair of breaks therein each extending through said waistband and through a lower edge of said outer covering such that each of said legs is divided approximately into a front portion and a back portion, a pair of couplers being mounted on said outer covering to releasably close said breaks.

5. The assembly according to claim 4, wherein each of said couplers comprises a hook and loop coupler extending approximately along an entire length of an associated one of said breaks.

6. An incontinence undergarment assembly comprising:
an inner covering configured for positioning against genitals of a person, said inner covering including a panel being flexible and having an inner surface and an outer surface, said panel having a front edge, a rear edge, a first side edge and a second side edge, each of said first and second side edges being concavely arcuate, said panel being formable into a wearable position such that said inner surface is concavely arcuate from said front edge to said rear edge, a central portion positioned between said front and rear edges comprising a crotch area, a lower most point of said outer surface of said crotch area defining a base of said inner covering;
an absorbent pad being mounted on said inner surface, said absorbent pad being spaced from said front and rear edges and being positioned on said crotch area;
an outer covering comprises a pair of shorts, said outer covering being positionable around said inner covering when said inner covering is in said wearable position such that a juncture of said first side edge and said front edge remains spaced from a juncture of said first side edge and said rear edge, and such that a juncture of said second side edge and said front edge remains spaced from a juncture of said second side edge and said rear edge, said outer covering completely covering said inner covering such that said inner covering is not viewable from a front side of said outer covering, said outer covering being removably coupled to said inner covering, said outer covering including a perimeter wall having an upper edge defining a waistband, said perimeter wall extending downwardly from said waist band and being formed into a pair of legs, each of said legs extending downwardly below said inner covering a distance between 2.0 inches and 12.0 inches, said outer covering including said front side and said back side, said outer covering including a pair of breaks therein each extending through said waistband and through a lower edge of said outer covering such that each of said legs is divided approximately into a front portion and a back portion;
a pair of couplers, each of said breaks having an edge having one of said couplers attached thereto, each of said couplers comprising a hook and loop coupler extending approximately along an entire length of an associated one of said breaks; and
wherein said outer covering can be detached from said inner covering and said inner covering replaced if said inner covering becomes soiled.
